(19) United States
(12) Patent Application Publication
(10) Pub. No.: US 2023/0064292 A1
(43) Pub. Date: Mar. 2, 2023

(54) METHODS OF TREATING PSMA-POSITIVE CANCER USING RADIONUCLIDE THERAPY

(71) Applicant: Cancer Targeted Technology LLC, Woodinville, WA (US)

(72) Inventors: Beatrice Langton-Webster, Woodinville, WA (US); Hillarie P. Windish, Woodinville, WA (US)

(21) Appl. No.: 17/759,754

(22) PCT Filed: Jan. 29, 2021
(86) PCT No.: PCT/US2021/015872

§ 371 Date: Jul. 29, 2022

Related U.S. Application Data
(60) Provisional application No. 62/967,553, filed on Jan. 29, 2020.

(51) Int. Cl. A61K 51/04; A61K 51/08; A61P 35/00

(52) U.S. Cl. CPC A61K 51/0497; A61K 51/088; A61P 35/00

(57) ABSTRACT

The invention relates to methods of treating cancer with CTT1403 having specificity for prostate-specific membrane antigen (PSMA).

### CROSS-REFERENCE TO RELATED APPLICATIONS

This application is a U.S. national phase of International Application No. PCT/US2021/015872, filed Jan. 29, 2021, which claims priority to U.S. Provisional Application No. 62/967,553, filed Jan. 29, 2020, the disclosure of each which is incorporated herein by reference in its entirety.

### STATEMENT OF GOVERNMENT RIGHTS

This invention was made with government support under grant number R44CA239461 awarded National Cancer Institute. The government has certain rights in the invention.

### BACKGROUND OF THE INVENTION

### Field of the Invention

The invention relates to methods of treating cancer with certain radiotherapeutics having specificity for prostate-specific membrane antigen (PSMA).

### Description of the Related Art

Prostate cancer is the most commonly diagnosed cancer and second leading cause of cancer death in American men. Hormone ablation or androgen deprivation therapy is widely used in the treatment of prostate cancer, but prostate cancer eventually becomes hormone resistant, rendering hormone ablation therapy ineffective. Prostate cancer is radiosensitive and, when localized, responds well to external beam radiation and brachytherapy. Despite this sensitivity, only a single radiotherapy is currently approved for treating metastases from prostate cancer. Radium-223 chloride (Xofigo®), targeting bone metabolism and cell turnover, was approved by the United States Food and Drug Administration (FDA) in 2013 for palliative treatment of bone metastases in prostate cancer, and results in an average of 3-4 months increase in survival. The modest survival advantage from radium-223 therapy can primarily be attributed to lack of specific targeting to prostate cancer sites and its inability to be used for soft tissue metastases. Specific targeting of the radiation payload to metastatic sites in advanced and widely disseminated prostate cancer is expected to improve efficacy and minimize side effects to non-target tissues.

Prostate-specific membrane antigen (PSMA) is a transmembrane protein also known as glutamate carboxypeptidase II (GCPII). PSMA is overexpressed on most prostate cancer cells as well as in the neovasculature of a variety of solid tumors. As a result, PSMA has attracted attention as a clinical biomarker for detection and management of prostate cancer. Generally, these approaches utilize an antibody specifically targeted to PSMA to direct imaging or therapeutic agents. For example, ProstaScint (Cytogen, Philadelphia, Pa.), which has been approved by the FDA for the detection and imaging of prostate cancer, utilizes an antibody to deliver a chelated radioisotope (Indium-111). However, it is now recognized that the ProstaScint technology is limited to the detection of non-viable cells and therefore its clinical relevance is questionable. Accordingly, it remains a challenge to develop effective methods and therapies to deliver PSMA-targeted small molecules to cancerous tissue.

### SUMMARY OF THE INVENTION

The inventors have discovered methods of effectively delivering PSMA-specific radiotherapeutic compounds to a patient in need thereof.

Accordingly, in one aspect, the present disclosure provides a method for treating cancer in a subject in need thereof, wherein the cancer has tumors that express PSMA or vasculature associated with the tumors expresses PSMA, the method comprising administering to the subject an effective amount of CTT1403 or a salt thereof, wherein CTT1403 is:

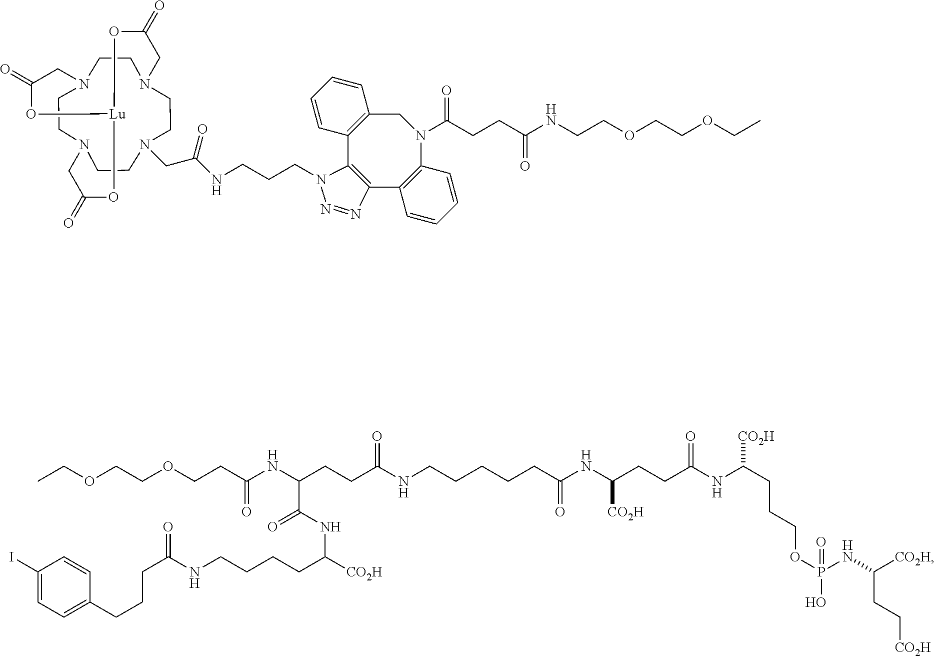

or a salt thereof, and
wherein the effective amount is about 0.75 GBq to about 14.0 GBq administered in 1 to 8 cycles.

In another aspect, the present disclosure provides a method for treating prostate cancer in a subject in need thereof, the method comprising:

identifying a subject as having PSMA-positive prostate cancer; and

administering to the subject a dose of about 0.75 GBq to about 14.0 GBq of CTT1403 or a salt thereof 1-8 times, wherein CTT1403 is:

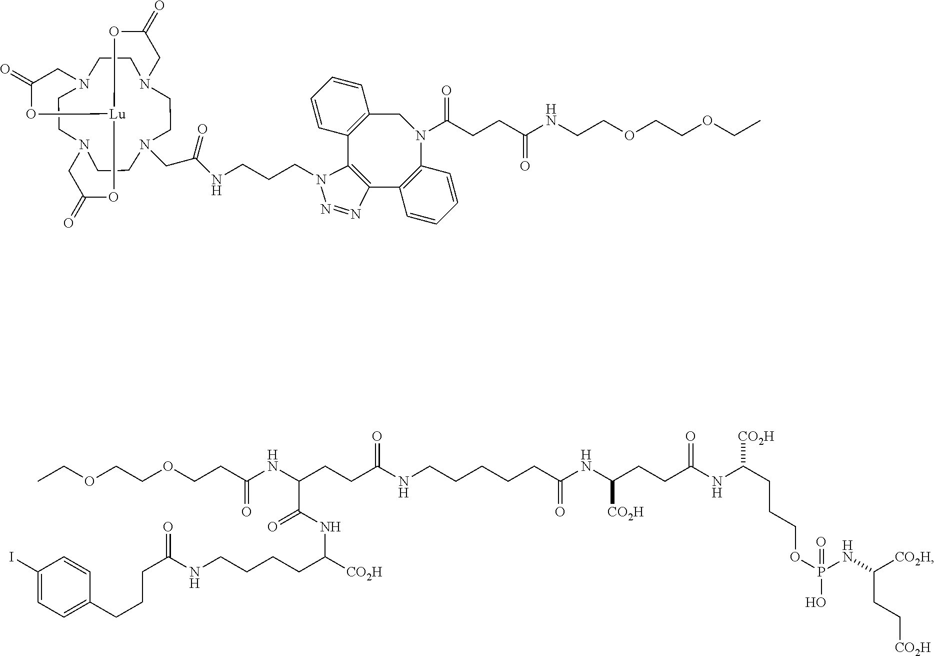

or a salt thereof.

In another aspect, the present disclosure provides for a method of administering at least about 10 Gy of absorbed radiation to the cancerous tissue of a patient being treated for cancer, comprising administering one or more doses of CTT1403 to the patient, where each dose comprises from about 6-12 GBq of radioactivity.

In another aspect, the present disclosure provides for a method of treating cancer in a patient having PSMA-positive tumors, comprising administering an amount of CTT1403 or a salt thereof effective to deliver at least about 10 Gy of absorbed radioactive dose to at least one of the PSMA positive tumors.

In another aspect, the present disclosure provides pharmaceutical formulations comprising CTT1403.

### BRIEF DESCRIPTION OF THE DRAWINGS

FIG. 1 displays the cellular uptake of CTT1403 compared to ¹⁷⁷Lu-PSMA-617 according to an example embodiment.

FIG. 2-8 show positron-emission tomography (PET) and single-photon emission computed tomography SPECT/CT images according to Example 4.

FIG. 9 displays SPECT and PET imaging of a patient (patient 12) according to an example embodiment. Restaging PET demonstrates some resolved tumors and some tumors with increased PSMA avidity. The arrows in the SPECT point to uptake of CTT1403 in tumors, even 7 days after dose administration. Dose level: 4.5 GBq.

FIG. 10 displays SPECT and PET imaging of a patient (patient 17) according to an example embodiment. Within 24 hours of administration of 7.5 GBq of CTT1403, distinct uptake of CTT1403 can be visualized in tumor. Dose level: 7.5 GBq.

### DETAILED DESCRIPTION OF THE INVENTION

Before the disclosed methods are described, it is to be understood that the aspects described herein are not limited to specific embodiments, or compositions, and as such can, of course, vary. It is also to be understood that the terminology used herein is for the purpose of describing particular aspects only and, unless specifically defined herein, is not intended to be limiting.

As used herein, the phrase “vasculature associated with the tumors” refers to blood and lymphatic vasculature adjacent and/or within a tumor and necessary for tumor viability and growth.

In view of the present disclosure, the methods described herein can be configured by the person of ordinary skill in the art to meet the desired need. In general, the disclosed methods provide improvements in the treatment of cancer.

One aspect of the present disclosure provides methods for treating cancer in a subject in need thereof. Such methods include administering to the subject an effective amount of CTT1403, or a salt thereof. CTT1403 may be prepared as known in the art, for example as disclosed in International Patent Application no. PCT/US2017/046352, incorporated herein by reference in its entirety.

In the present disclosure CTT1403 refers to a ¹⁷⁷Lu chelate having the structure

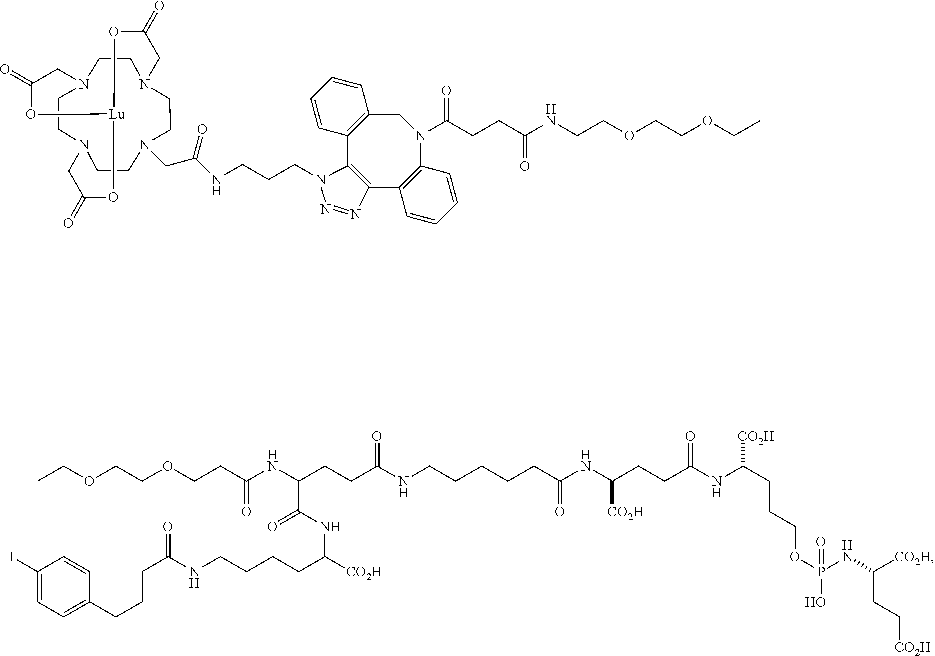

and can be named ¹⁷⁷lutetium (4S,9S)-1-(((((S)-1,3-dicarboxypropyl)amino)(hydroxy)phosphoryl)oxy)-21-(17,20-dioxo-20-(1-(3-(2-(3,16,19-trioxo-2,17,18-trioxa-5,8,11,14-tetraazatricyclo[9.6.3.2^{5,14}]docosan-8-yl)acetamido)propyl)-1H-dibenzo[b,f][1,2,3]triazolo[4,5-d]azocin-8(9H)-yl)-4,7,10,13-tetraoxa-16-azaicosanamido)-33-(4-iodophenyl)-6,11,18,22,30-pentaoxo-5,10,17,23,29-pentaazatritriacontane-4,9,24-tricarboxylic acid, or a salt thereof.

Thus, CTT1403 itself is a chelate compound comprising ¹⁷⁷Lu coordinated to a larger molecule. Accordingly, it will be understood that CTT1403 as referred to in the present disclosure may be present as a salt, wherein one or more acid moieties (e.g., carboxylic acid) are deprotonated, and/or one or more amine or basic moieties are protonated. Suitable cations and anions are readily apparent to one of skill in the art and include pharmaceutically-acceptable salts. For example, CTT1403 may be provided as the sodium salt (e.g., the pentasodium salt). In an embodiment, CTT1403 is present as the sodium salt and can be named 1,5-disodium 2-[({[4-(4-{6-[4-({6-[4-(4-iodophenyl)butanamido]-1-oxo-1-(sodiooxy)hexan-2-yl}carbamoyl)-4-(1-{4-oxo-4-[3-(3-{2-[3,16,19-trioxo(1-¹⁷⁷Lu)-2,17,18-trioxa-5,8,11,14-tetraaza-1-lutetatricyclo[9.6.3.2^{5,14}]docosan-8-yl]acetamido}propyl)-3,4,5,13-tetraazatetracyclo[13.4.0.0^{2,6}.0^{7,12}]nonadeca-1(15),2(6),4,7(12),8,10,16,18-octaen-12-yl]butanamido}-3,6,9,12-tetraoxapentadecan-15-amido)butanamido]hexanamido}-5-oxo-5-(sodiooxy)pentanamido)-5-oxo-5-(sodiooxy)pentyl]oxy}(sodiooxy)phosphoryl)amino]pentanedioate.

A drawback of conventional radionuclide drugs is their low circulation half-life, leading to rapid elimination from the body and/or kidney accumulation. CTT1403 advantageously includes an albumin binding motif that is designed to increase circulation time. This is hypothesized to both reduce kidney accumulation, which is often the source of dose-limiting toxicity, and increase the proportion of drug that becomes associated with a tumor and/or cancerous tissue, such as the tumor vasculature. This will increase effectiveness and/or allow for lower dosing with a similar result, leading to enhanced patient outcomes.

In certain embodiments as otherwise described herein, the cancer is prostate cancer (e.g., prostate adenocarcinoma) in certain embodiments, the patient will not have de novo small cell carcinoma of the prostate. Without wishing to be bound by theory, it is presently believed that CTT1403 or a salt thereof, as well as certain imaging agents, bind to PSMA. Accordingly, the methods of the present disclosure are especially useful in cancers that include tumors with PSMA expression on the surface of the tumor. Such cancers are termed PSMA-positive (PSMA+), or may also be referred to as PSMA-avid. PSMA-positive cancers can be determined using a number of methods known to one of skill in the art. Such methods include imaging (e.g., PET imaging, single-photon emission computed tomography (SPECT) imaging), immunohistochemistry, or biochemical assessment. In certain embodiments as otherwise described herein, the subject is identified as having PSMA-positive cancer (e.g., PSMA-positive prostate cancer). In particular embodiments as otherwise described herein, the effective amount of CTT1403 to be administered in an initial or subsequent treatment cycle (optionally spaced by a treatment interval), is determined using single-photon emission computed tomography (SPECT) dosimetry. In certain embodiments, the method further comprises imaging cancer using single-photon emission computed tomography (SPECT) dosimetry; and determining the effective amount of CTT1403 based on the SPECT imaging data. For example, in certain embodiments, the frequency or length of a particular treatment cycle is determined using pharmacokinetic analysis of CTT1403 blood levels. The method as otherwise described herein, may, in certain embodiments, further comprise analyzing CTT1403 blood levels; and determining frequency or length administration of CTT1403 based on the CTT1403 blood levels.

An alternative approach to monitoring disease progression is PSA levels, as commonly known in the art. Accordingly, in certain embodiments as otherwise described herein, the method further comprises measuring PSA levels; and determining frequency or length administration of CTT1403 and/or the effective amount of CTT1403 based on the measured PSA-levels.

The diagnostic methods as disclosed herein may be used individually or in combination as needed.

¹⁷⁷Lutetium is a radioactive nuclide that exhibits a radioactive half-life of approximately 6.6 days. The beta particle emitted from the radioactive decay of ¹⁷⁷Lu can damage tissue, leading to an anticancer effect if localized in cancerous tissue. The gamma particle emitted also allows for SPECT imaging to assess dosimetry to tumors and other tissues. Without wishing to be bound by theory, it is believed that CTT1403 or a salt thereof efficiently binds to prostate-specific membrane antigen (PSMA) through a PSMA-binding motif. As some cancerous tumors, including prostate cancer tumors, upregulate PSMA expression, this binding leads to concentration of CTT1403 in cancerous tumors, allowing increased localization of radioactivity leading to tissue damage. PSMA-positive prostate cancer is known to express PSMA on the surface of the tumor and is thus a preferential candidate for treatment with CTT1403 or a salt thereof.

Anti-androgen treatment is a common therapy for treating prostate cancer. In some cases, cancerous tissues depend on sufficient androgen levels and androgen suppression effectively halts disease progression. In some cases, however, the anti-androgen treatment is not effective, and is termed castration resistant. Thus, in certain embodiments described herein, the prostate cancer is castration resistant, and/or the target tumor is castration resistant. Castration resistant prostate cancers are those that continue to progress despite androgen depletion therapy. Increased PSMA expression has been correlated with metastatic progression and castration resistance. As such, castration resistant prostate cancer is an attractive target for PSMA-targeted therapies. Anti-androgen treatment may also lead to increased PSMA expression. Accordingly, in certain embodiments, the subject previously received an anti-androgen treatment. For example, CTT1403 or a salt thereof can be administered after the subject has received an anti-androgen treatment. Such treatment can be in the form of androgen synthesis inhibitors, androgen receptor antagonists, or combinations thereof. Examples of anti-androgen treatments include androgen receptor inhibitors such as enzalutamide, nilutamide, flutamide, or bicalutamide, nonspecific steroidal biosynthesis inhibitors such as ketoconazole, itraconazole, or abiraterone, and steroids such as prednisone, diethylstilbesterol, or dexamethasone. Other anti-androgen therapies are known in the art. Accordingly, a preferred patient population may include subject who are on at least one anti-androgen treatment, such an androgen signaling inhibitor. Subjects may or may not have a history of orchiectomy. Subjects without a history of bilateral orchiectomy may be required to remain on a luteinizing hormone-releasing hormone (LHRH) analog during the course of therapy.

In some aspects, cancers and/or the tumors thereof respond favorably to anti-androgen treatment and are considered to be androgen sensitive. These tumors also express PSMA and CTT1403 is believed to be effective in the treatment of such androgen sensitive tumors and cancers. Thus, in certain other embodiments, the prostate cancer is androgen sensitive, and/or the target tumor is androgen sensitive. In other embodiments, the prostate cancer has not been tested for androgen sensitivity.

The PSMA-positive cancer may be a localized cancer, wherein cancerous tissue is predominantly located in a particular part of the body, or predominantly located in one organ. In certain embodiments as otherwise described herein, the cancer is localized. In other cases, or after disease progression, the cancer may be a metastatic cancer, where cancerous tissue is found in several parts of the body and/or several organs. In certain embodiments as otherwise described herein, the cancer is metastatic. The PSMA-positive cancer may also be an organ specific cancer. It may be the case that the cancer is organ specific but has escaped the primary organ or origin. In particular embodiments, the cancer is organ specific.

The methods of the disclosure, in certain embodiments, further include administering an imaging agent to the subject. A variety of imaging agents is known in the art and can be used diagnostically to visualize cancerous tissue and monitor therapy progress. Examples of useful imaging agents include, but are not limited to, CTT1057, ¹⁸F-DCFPyl, ¹⁸F-PSMA-1007, and ⁶⁸Ga-PSMA-11. As used herein, CTT1057 is

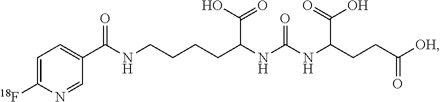 [SMILES: O=C(O)CCC(NC(=O)NC(CCCCNC(=O)c1ccc([18F])nc1)C(=O)O)C(=O)O]

and may be named (((S)-4-carboxy-4-((S)-4-carboxy-4-(6-(4-(fluoro-¹⁸F)benzamido)hexanamido)butanamido)butoxy)(hydroxy)phosphoryl)-L-glutamic acid, or a salt thereof. As used herein, ¹⁸F-DCFPyl is ((1-carboxy-5-(6-(fluoro-18F)nicotinamido)pentyl)carbamoyl)glutamic acid. As used herein, ¹⁸F-PSMA-1007 is ((1-carboxy-5-(2-(4-((4-carboxy-2-(4-carboxy-2-(6-(fluoro-18F)nicotinamido)butanamido)butanamido)methyl)benzamido)-3-(naphthalen-2-yl)propanamido)pentyl)carbamoyl)glutamic acid. As used herein, ⁶⁸Ga-PSMA-11 is 22-(3-(((2-((5-(2-carboxyethyl)-2-hydroxybenzyl)(carboxymethypamino)ethyl)(carboxymethyl)amino)methyl)-4-hydroxyphenyl)-5,13,20-trioxo-4,6,12,19-tetraazadocosane-1,3,7-tricarboxylic acid ⁶⁸Ga salt. For example, in certain embodiments, the imaging agent is CTT1057. In other embodiments, the imaging agent is ⁶⁸Ga-PSMA-11. In some embodiments, an imaging agent is administered prior to the administering of CTT1403 or a salt thereof. This allows for finding of the location of the tumor and, if a PSMA-binding imaging agent is used, can provide an indication as to the likely success of therapy. In certain embodiments, the imaging agent is administered after the administering of CTT1403 or a salt thereof. Administration of the imaging agent after therapy can be useful in determining the efficacy of treatment. In some embodiments, the imaging agent is administered both prior to and after the administering of CTT1403 or a salt thereof. This approach allows for comparison of the localization and size of cancerous tissue, allowing determination of therapy efficacy and informing future treatment.

In certain embodiments as otherwise described herein, the method further includes imaging the subject. Imaging can be particularly effective after the administration of an imaging agent as discussed above, and can be used to enhance therapeutic efficacy of the prostate cancer treatments as otherwise described herein.

In certain embodiments as otherwise described herein, CTT1403 or a salt thereof is administered on a dosage schedule, wherein the dosage schedule includes 1 to 8 treatment cycles, e.g., 1-6 treatment cycles, or 4-6 treatment cycles. For example, in certain embodiments, CTT1403 or a salt thereof is administered on a dosage schedule, wherein 2,3, 4,5,6,7, or 8 doses are administered at an interval of from about 1 to 16 weeks between each dose. The interval may be adjusted according to the needs of the patient. For example, in certain embodiments, the interval between doses is about 3 to 12 weeks, or about 4 to 8 weeks, or about 4 weeks, or about 6 weeks, or about 8 weeks. In particular embodiments, each interval constitutes a treatment cycle (e.g., one treatment cycle is complete after the interval has elapsed). CTT1403 or a salt thereof may be administered once at the beginning of each treatment cycle. Given the relatively short half-life of ¹⁷⁷Lu, re-administration of CTT1403 or a salt thereof may be desired to replenish or otherwise increase the radioactive dose afforded to the cancerous tissue. The dosage of each cycle can be the same as the initial dose, or can be reduced or increased as deemed necessary. The imaging results can be used to inform the dosage of each treatment cycle, whereby a change in the size or location of the cancerous tissue informs a change in the dosage. The time interval between dosages depends on the pharmacokinetics of the drug in the subject. In certain embodiments, the treatment cycle is about 28 days (e.g., the second dose is given 28 days after the initial dose). In other embodiments, the treatment cycle is about 30-60 days, or about 30-50 days, or about 30-40 days, or about 40-45 days, or about 42 days. In other embodiments, each treatment cycle is about 3-12 weeks, or about 4-8 weeks, or about 6 weeks. In certain embodiments, each treatment cycle is 2-6 weeks in duration, e.g., 2-4 weeks, or 3-4 weeks. In certain embodiments, each treatment cycle is 8-14 weeks in duration, e.g., 8-12 weeks, or 10-12 weeks. In other embodiments, the beginning of a new treatment cycle after 3-10 half-lives of ¹⁷⁷Lu have elapsed, or about 4-8 half-lives, or about 5 half-lives. In certain embodiments, CTT1403 or a salt thereof is administered once at the beginning of each treatment cycle. In other embodiments, CTT1403 or a salt thereof is administered in stages throughout the treatment cycle.

In another aspect, the present disclosure provides for a method of administering at least about 10 Gy of absorbed radiation to the cancerous tissue of a patient being treated for cancer, comprising administering one or more doses of CTT1403 to the patient, where each dose comprises from about 6-12 GBq of radioactivity. In certain embodiments as otherwise described herein, the cancerous tissue is PSMA-positive.

In another aspect, the present disclosure provides for a method of treating cancer in a patient having PSMA-positive tumors, comprising administering an amount of CTT1403 or a salt thereof effective to deliver at least about 10 Gy of absorbed radioactive dose to at least one of the PSMA positive tumors. In certain embodiments, the cancer is prostate cancer. In particular embodiments as otherwise described herein, the effective amount of CTT1403 is about 7 GBq to about 14 GBq, or about 7 GBq to about 12 GBq, or about 7 GBq to about 10 GBq. In particular embodiments, the effective amount is administered as a single dose.

In other embodiments, administration of CTT1403 of a salt thereof is followed by a period of monitoring of the prostate cancer. Monitoring can be done via PET, PSA screening, SPECT, RECIST 1.1 criteria, CT scan, MRI or PK analysis of blood levels. The RECIST 1.1 criteria are known in the art, for example disclosed by Eisenhauer et al., “New response evaluation criteria in solid tumors: Revised RECIST guideline (version 1.1)” Eur. J. Cancer, 45: 228-247 (2009). After a period of monitoring, another administration of CTT1403 or a salt thereof may be performed. An additional method to determine treatment effectiveness is through the measurement of tumor uptake of CTT1403. Accordingly, in certain embodiments the method further comprises measuring the uptake of CTT1403 within one or more of the cancer tumors and comparing the uptake to a predetermined threshold value. The threshold value would be apparent to one of skill in the art, and may be adjusted as treatment progresses.

The appropriate dosage of CTT1403 or a salt thereof can be expressed as units of Becquerel (e.g., gigabecquerel, GBq) in order to reflect the radioactivity of the ¹⁷⁷Lu. Accordingly, in certain embodiments, CTT1403 or a salt thereof is administered in an amount of about 0.5 GBq to about 14.0 GBq. For example, in certain embodiments, the effective amount of CTT1403 or a salt thereof is administered in an amount of about 0.75 GBq to about 14.0 GBq, e.g., in an amount of about 0.75 GBq to about 11.0 GBq, or about 0.75 GBq to about 10.0 GBq, or about 0.75 GBq to about 9.0 GBq, or about 0.75 GBq to about 8.0 GBq, or about 0.75 GBq to about 7.0 GBq, or about 0.75 GBq to about 6.0 GBq, or about 0.75 GBq to about 5.0 GBq, or about 0.75 GBq to about 4.0 GBq, or about 0.75 GBq to about 3.5 GBq, or about 0.75 GBq to about 3.0 GBq, or about 0.75 GB to about 2.0 GBq, or about 0.75 GBq to about 1.5 GBq, or about 0.75 GBq to about 1 GBq. In certain embodiments, the CTT1403 is administered in an amount of about 1.5 GBq to about 14.0 GBq, e.g., about 2.0 GBq to about 14.0 GBq, or about 3.0 GBq to about 12.0 GBq, or about 3.5 GBq to about 12.0 GBq, or about 4.0 GBq to about 12.0 GBq, or about 5.0 GBq to about 12.0 GBq, or about 6.0 GBq to about 12.0 GBq, or about 7.0 GBq to about 12.0 GBq, or about 8.0 GBq to about 12.0 GBq, or about 9.0 GBq to about 12.0 GBq, or about 10.0 GBq to about 12.0 GBq, or about 12.0 GBq to about 14.0 GBq. In certain embodiments, the CTT1403 is administered in an amount of about 1.5 GBq to about 10.0 GBq, e.g., about 1.5 GBq to about 9.0 GBq, or about 1.5 GBq to about 8.0 GBq, or about 1.5 GBq to about 7.0 GBq, or about 1.5 GBq to about 6.0 GBq, or about 1.5 GBq to about 5.0 GBq, or about 1.5 GBq to about 4.0 GBq, or about 1.5 GBq to about 3.5 GBq, or about 1.5 GBq to about 3.0 GBq. In other embodiments, CTT1403 or a salt thereof is administered in an amount of 0.75±10% GBq, 1.5±10% GBq, 2.0±10% GBq, 2.5±10% GBq, 3.0±10% GBq, 3.5±10% GBq, 4.0±10% GBq, 4.5±10% GBq, 5.0±10% GBq, 5.5±10% GBq, or 6.0±10% GBq, or 7.0±10% GBq, or 8.0±10% GBq, or 9.0±10% GBq, or 10.0±10% GBq, or 11.0±10% GBq, or 12.0±10% GBq, or 13.0±10% GBq, or 14.0±10% GBq. The GBq amounts can be readily converted to millicurie values by using the conversion factor of 0.037 GBq/1 mCi.

A sample of pure CTT1403 or salt thereof (i.e., without including any radioactive decay products) would have an activity of approximately 4,104 GBq per mg of ¹⁷⁷Lu (i.e., approximately 4.104 GBq per μg of ¹⁷⁷Lu). Accordingly, the above dosages can be converted to weight-based dosages based upon this specific activity and the molecular weight of CTT1403 or salt thereof. For example, in certain embodiments, CTT1403 or salt thereof is administered in an amount of about 0.12 μg of ¹⁷⁷Lu to about 3.41 μg of ¹⁷⁷Lu.

Each dose of CTT1403 is expected to administer an absorbed radiation to at least one of the tumors of the PSMA-positive caner in the patient in need thereof. In certain embodiments as otherwise described herein, each effective amount of CTT1403 delivers at least about 8 Gy of absorbed radiation to at least one cancer tumor, per tumor. For example, in certain embodiments, the absorbed radiation to the at least one cancer tumor is in the range of 8 Gy to 50 Gy, or 10 Gy to 50 Gy, or 12 Gy to 50 Gy, or 14 Gy to 50 Gy, or 16 Gy to 50 Gy, or 8 Gy to 40 Gy, or 10 Gy to 40 Gy, or 12 Gy to 40 Gy, or 14 Gy to 40 Gy, or 16 Gy to 40 Gy, or 8 Gy to 35 Gy, or 10 Gy to 35 Gy, or 12 Gy to 35 Gy, or 14 Gy to 35 Gy, or 16 Gy to 35 Gy, or 8 Gy to 30 Gy, or 10 Gy to 30 Gy, or 12 Gy to 30 Gy, or 14 Gy to 30 Gy, or 16 Gy to 30 Gy, or 12 Gy to 25 Gy, or 14 Gy to 25 Gy. In particular embodiments, about 10 to about 25 Gy, or about 10 Gy to about 16 Gy, or about 10 Gy to about 14 Gy of absorbed radiation are administered. In particular embodiments, at least one tumor receives an absorbed radiation of at least 20 Gy, or at least 22 Gy. While the upper limit on absorbed radiation is not essential and is limited by other toxicity criteria, an upper limit may be taken as 50 Gy, or 40 Gy.

In particular embodiments, each effective amount of CTT1403 delivers at least about 8 Gy to at least two tumors, or at least three tumors, associated with the PSMA-positive cancer.

In certain embodiments as otherwise described herein, CTT1403 or a salt thereof is delivered in a radiostabilizing solution. Radiostabilizing solutions include chemicals that prevent, reduce, or repair damage caused by the radionuclide. This allows additional active pharmaceutical to be maintained through transport and storage of the formulation, and also reduces the presence of potentially harmful degradation products. A number of radiostabilizing chemicals are known in the art, including amino acids (e.g., L-methionine, tryptophan, cysteine, cysteine ethyl ether, histidine, glycine), reducing agents (mercaptoethanol, dithiothreitol), selenocysteine, selenomethionine, gentisic acid and ascorbic acid, or salts thereof (e.g., sodium gentisate, sodium ascorbate), human serum albumin, and ethanol. Combinations of two or more radiostabilizers can be used. In an embodiment, the radiostabilizing solution comprises sodium gentisate, sodium ascorbate, and L-methionine. The radiostabilizers are generally provided in concentrations that maximize their effectiveness without causing other deleterious effects. For example, the radiostabilizers can be present in amount of 1-20 mM, or 2-16 mM (e.g., 2-12 mM, or 2-10 mM, or 2-8 mM, or 2-6 mM, or 2-4 mM, or 4-8 mM, or 4-12 mM). In an embodiment, the radiostabilizing solution includes 2-6 mM (e.g., 4 mM) sodium gentisate, 2-8 mM (e.g., 6 mM) sodium ascorbate and 2-6 mM (e.g., 4 mM) L-methionine. The radiostabilizing solution can also be a saline solution, and can include ethanol (e.g., up to 8% ethanol).

### Pharmaceutical Compositions

In some embodiments, the method comprises the administration of CTT1403 and/or the imaging agent in a pharmaceutical composition having at least one pharmaceutically acceptable carrier, excipient, solvent, adjuvant or diluent.

The small molecules described herein may be administered parenterally in formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

The drug compositions disclosed herein may be administered intravenously. For example, CTT1403 or a salt thereof may be prepared in a 25-200 MBq/mL solution and diluted to a final volume of 1-100 mL for administration.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

### Definitions

As used in the specification and the appended claims, the singular forms a, “an” and “the” include plural referents unless the context clearly dictates otherwise.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivative or pro-drug, such as pharmaceutically acceptable salts and esters. The manufacture of pharmaceutical compositions for different routes of administration falls within the capabilities of a person skilled in medicinal chemistry.

### EXAMPLES

The methods of the disclosure are illustrated further by the following examples, which are not to be construed as limiting the invention in scope or spirit to the specific procedures described in them.

In the Examples below, CTT1403 was formulated in a solution of 4 mM gentisic acid, 6mM sodium ascorbate, and 4 mM L-methionine in saline, further containing up to 8% ethanol.

### Example 1

In order to investigate the pharmacokinetics of CTT1403, doses were administered by IV to fifteen subjects. The subjects each had histologically confirmed prostate adenocarcinoma that was metastatic and castration resistant (mCRPA). Subjects with de novo small cell carcinoma of the prostate were excluded. The subjects further had at least three metastatic foci avid for PSMA-specific PET agent CTT1057 or 68Ga-PSMA-11 uptake. PSMA avidity was defined as SUVₘₐₓ>mediastinal blood pool. The subjects had received docetaxel, were ineligible for docetaxel, or had refused docetaxel. They all further had progression by the PCWG3 criteria during or after treatment with abiraterone, enzalutamide, and/or apalutamide, and had serum testosterone<50 ng/dL. Subjects without a history of bilateral orchiectomy were required to remain on a luteinizing hormone-releasing hormone (LHRH) analog during the course of the protocol therapy. Subjects were excluded if: they had received treatment with radium-223 or another radiopharmaceutical within 3 months prior to the first dose of CTT1403; they had received prior systemic anti-cancer therapy (excluding radiopharmaceutical) within 14 days, or 5 half-lives, whichever is shorter, prior to the first dose of CTT1403, they had received external-beam radiation within 14 days prior to the first dose of CTT1403; they had received cabazitaxel for the treatment of mCRPC; they had received previous treatment with a therapeutic targeting PSMA; they had a major surgical procedure 28 days before the first dose of CTT1403. Subjects were eligible for a second treatment cycle in the event that they exhibited an absence of clinical progression or radiographic progression by PGWC3 criteria following the first dose of CTT1403, and had adequate organ function.

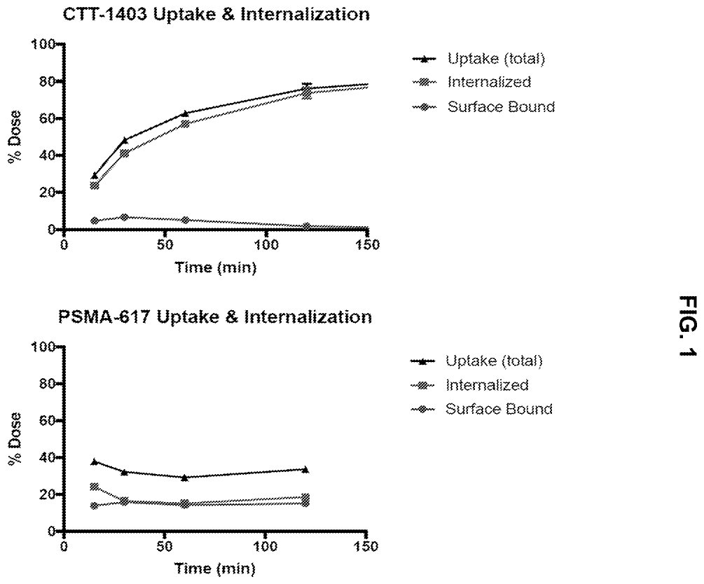

The PSMA binding scaffold of PSMA binds irreversibly to the extracellular enzymatic domain of PSMA leading to rapid internalization by PSMA-expressing cancer cells. Tumor cell uptake of CTT1403 is ˜84% after just 2½ hours (FIG. 1), compared to less than 40% tumor cell uptake of ¹⁷⁷Lu-PSMA-617 (structure shown below), in PSMA-expressing PC3-PIP cells.

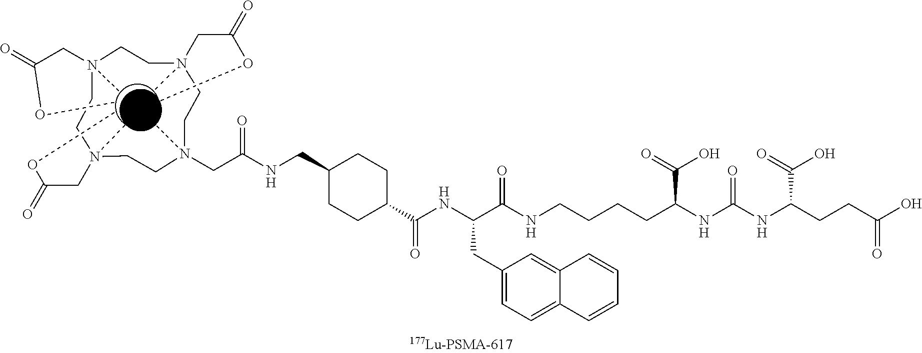

Given that 99% of cell bound CTT1403 is internalized, compared to just 15% of ¹⁷⁷Lu-PSMA-617, targeting by CTT1403 is expected to confer a significant therapeutic advantage. This greater uptake will allow treatment of smaller tumors for a longer time. Both irreversible binding and cellular internalization increases exposure of target tissue to the therapeutic agent. Greater accumulation in the target tissue will permit a lower efficacious dose and/or dosing frequency, thus reducing a patient's total clinical exposure to the radionuclide and improving the therapeutic index.

Pharmacokinetics assessment follows the movement of drugs throughout the body, including measurement of the drug's half-life in circulation. The longer a drug remains available in circulation before being cleared from the body, the greater the opportunity for it to meet its target and achieve the desired therapeutic effect. To measure pharmacokinetics, remaining CTT1403 was estimated by measuring the radioactivity of blood sample withdrawn at particular time points. Each patient received a dose ranging from 0.75 GBq to 7.5 GBq. Even after 2 weeks, CTT1403 was still detected in subjects who received the 7.5 GBq dose. The derived pharmacokinetic data is shown in Table 1:

TABLE 1

| | Distribution | Elimination |
|---|---|---|
| | Half-life | Half-life 2 |
| Dose | (h) | (h) |
| 1.5 GBq (1^{st} patient at this dose) | 0.75 | 28.88 |
| 2.0 GBq (1^{st} patient at this dose) | 1.04 | 23.90 |
| 3.0 GBq (1^{st} patient at this dose) | 0.51 | 27.73 |
| 3.0 GBq (2^{nd} patient at this dose) | 0.89 | 31.51 |
| 3.0 GBq (3^{rd} patient at this dose) | 0.70 | 36.48 |
| 4.5 GBq (1^{st} patient at this dose) | 0.85 | 33.01 |
| 4.5 GBq (2^{nd} patient at this dose) | 0.85 | 34.66 |
| 4.5 GBq (3^{rd} patient at this dose) | 0.85 | 43.32 |
| 6.0 GBq (1^{st} patient at this dose) | 0.78 | 46.21 |
| 6.0 GBq (2^{nd} patient at this dose) | 0.57 | 30.14 |
| 6.0 GBq (3^{rd} patient at this dose) | 1.06 | 33.01 |
| 7.5 GBq (1^{st} patient at this dose) | 1.20 | 57.76 |
| 7.5 GBq (2^{nd} patient at this dose) | 1.00 | 57.76 |

| | |
|---|---|
| Distribution half-life (h) | 0.85 |
| Standard Deviation | 0.20 |
| Elimination half-life (h) | 37.26 |
| Standard Deviation | 10.90 |

Pharmacokinetic evaluation demonstrated that CTT1403 circulation has a preliminary biphasic half-life of 0.85±0.2 hours (h) (range: 0.51-1.2 h) for the distribution phase and 37.3±10.9 h (range: 0.2-57.8 h) for the elimination phase. For comparison, ¹⁷⁷Lu-PSMA-617 has a reported initial half-life of 0.16±0.09 hours during the distribution phase, and a half-life of 10.8±2.5 hours during the elimination phase. This is already more than a 5.3-fold greater distribution phase and 3.5-fold greater elimination phase than the PSMA-617 agent (without the albumin binder) and will further increase as the dose increases. CTT1403 has an at least two to ten-fold greater circulation time than the ¹⁷⁷Lu-PSMA-617 agent which lacks the albumin binding moiety of CTT1403. An increased circulation time as observed in the preclinical studies is correlated with efficacy and significantly increased survival advantage. Taken together, CTT1403 demonstrates prolonged blood circulation, low dose limiting organ exposure and extended tumor exposure have been observed early in the first-in-human clinical trial of CTT1403 in mCRPC patients. CTT anticipates that these early observations will translate to compelling efficacy as the trial progresses. Table 1 reports the blood distribution and elimination half-lives for 13 of 15 patients enrolled in the study. The derived clear half-lives are at least two to ten-fold greater than the PSMA-617 agent which lacks an albumin binder. Longer clearance half-lives will advantageously yield enhanced drug delivery as CTT1403 has more time to find and bind to expressed PSMA. This effect also allows more time for the therapeutic agent to reach smaller tumors or poorly-vascularized tumors. Longer systemic circulation of CTT1403 afforded by the albumin motif believed to lead to lower dosing of the kidneys, a dose-limiting factor, allowing higher dosages to be safely administered.

### Example 2

CTT1403 localization in various organs is accomplished by estimating dosimetry using SPECT/CT or SPECT/MRI imaging of the radioisotope component (using the gamma particle emission) of CTT1403, ¹⁷⁷Lu, up to 8 days after the first dose. Previous studies of dosimetry in patients administered ¹⁷⁷Lu-PSMA-617 have identified salivary glands, kidneys and bone marrow as potential dose limited organs. Preclinical studies in rats with CTT1403 identified the kidneys as the dose limiting organ. Table 2, below, summarizes kidney dosimetry calculated from the first 15 patients enrolled in the study after receipt of escalating doses of CTT1403 and contrasts it with the predicted kidney exposure from rat studies. Due to the known overexpression of PSMA in rat kidneys, the predicted kidney dose was conservatively overestimated, and the radiation exposure of human kidney has been demonstrated to be significantly less than predicted. Preclinical studies in rats estimated expected kidney exposure of ˜5.2 Gy/GBq. Kidney dosimetry calculated from the first 15 patients enrolled in the study is lower by ˜ a factor of 10: 0.687 Gy/GBq, significantly less than predicted by the rat studies. This may be due, in part, to the overexpression of PSMA in rodent kidney, but that does not entirely explain the ˜10× reduced kidney dose observed clinically. It has been demonstrated clinically that dose of another PSMA-targeting agent reaches unaffected tissues less with increasing tumor burden. CTT1403 may be sequestered in tumors and under exposing the kidney.

TABLE 2

| | | | | | absorbed | absorbed |
|---|---|---|---|---|---|---|
| | injected | injected | absorbed | absorbed | dose per GBq | dose per GBq |
| | activity | activity | dose (Gy) - | dose (Gy) - | (Gy/GBq) - | (Gy/GBq) - |
| Patient | (GBq) | (mCi) | L. kidney | R. kidney | L. kidney | R. kidney |
| 1 | 0.73 | 19.6 | 0.441 | 0.430 | 0.608 | 0.593 |
| 2 | 1.50 | 40.5 | 0.915 | 0.876 | 0.610 | 0.584 |
| 3 | 2.03 | 54.8 | 1.54 | 2.07 | 0.757 | 1.020 |
| 4 | 3.00 | 81.1 | 1.97 | 1.70 | 0.655 | 0.565 |
| 5 | 2.95 | 79.8 | 4.41 | 4.84 | 1.494 | 1.638 |
| 6 | 2.98 | 80.5 | 2.75 | 2.65 | 0.922 | 0.889 |
| 7 | 4.48 | 121.1 | 2.54 | 2.83 | 0.567 | 0.632 |
| 8 | 4.52 | 122.2 | 3.45 | 3.54 | 0.764 | 0.784 |
| 9 | 3.53 | 95.5 | 1.71 | 1.85 | 0.484 | 0.522 |
| 10 | 4.48 | 121.2 | 2.26 | 2.75 | 0.505 | 0.612 |
| 11 | 5.66 | 153.0 | 3.32 | 3.08 | 0.587 | 0.543 |
| 12 | 5.99 | 162.0 | 3.15 | 2.81 | 0.525 | 0.469 |
| 13 | 5.99 | 162.0 | 2.65 | 2.50 | 0.443 | 0.417 |
| 14 | 7.98 | 215.7 | 5.53 | 5.41 | 0.693 | 0.678 |
| 15 | 7.77 | 210.0 | 4.89 | 3.75 | 0.629 | 0.482 |

### Example 3

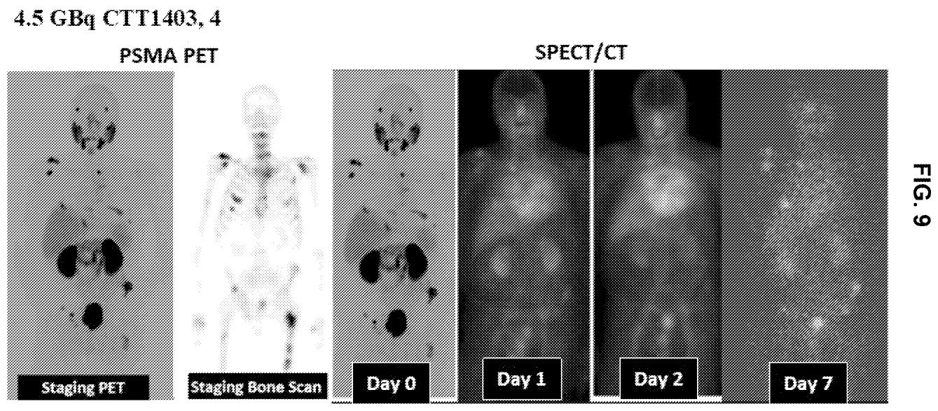

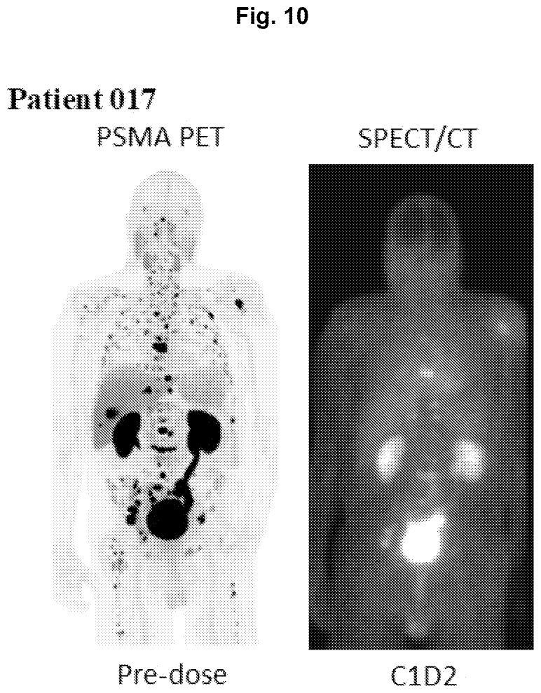

In addition to assessing radiation exposure to dose limiting organs, it is also feasible to use SPECT/CT or SPECT/MRI imaging and dosimetry to estimate CTT1403 dose to the tumor, which could indicate efficacy. FIG. 9 similarly shows that CTT1403 is detected by SPECT within a tumor that later had diminishing PSMA avidity in a patient who received 4.5 GBq. In addition, at 4.5 GBq, the time course of SPECT imaging demonstrates visibly increasing levels of CTT1403 detected within tumor over time, and that CTT1403 is still clearly present within tumor through 7 days post-administration. Finally, FIG. 10 demonstrates distinct uptake of CTT1403 in tumors just 24 hours after administration of 7.5 GBq CTT1403, suggesting that tumor is exposed at a high level earlier following CTT1403 administration compared to lower doses. As shown in Table 3, below, preliminary analysis of CTT1403 uptake into PSMA+ tumors following a single 7.5 GBq dose demonstrated tumor doses of 9.74-22.40 Gy resulting from the single dose of CTT1403.

Using CTT1057 PET imaging, specific PSMA+tumors can be identified, examined for CTT1403 uptake using SPECT, then re-examined in a follow up CTT1057 PET image to demonstrate whether uptake of CTT1403 was associated with regression of the tumor. Published studies using ¹⁷⁷Lu-PSMA-617 suggest that delivery of 10-14 Gy over several cycles of therapy is associated with efficacy based on reduction of circulating prostate specific antigen (PSA), a measure of biochemical recurrence. Table 3 demonstrates that dose to the tumor increases with administered CTT1403 dose and that following administration of a single dose of about 7.5 GBq CTT1403, some tumors can be seen to absorb 9.7-2.4 Gy, a significant increase over ˜1 Gy administered by 3.0 GBq. These data suggest that increasing the dose of CTT1403 to up to 14.0 GBq will increase radiation exposure to the tumor with each administered dose.

TABLE 3

| | Dose | Dose | Tumor 1 | Tumor 2 | Tumor 3 |
|---|---|---|---|---|---|
| Patient | (GBq) | (mCi) | (Gy) | (Gy) | (Gy) |
| 1 | 0.73 | 19.6 | 0.12197 | 0.11641 | |
| 2 | 1.5 | 40.5 | 0.24610 | | |
| 3 | 2.03 | 54.8 | | 0.25532 | |
| 4 | 3.0 | 81.1 | 1.28165 | 0.37288 | 1.90649 |
| 5 | 2.95 | 79.8 | 1.63929 | | |
| 6 | 2.98 | 80.5 | 1.10352 | 0.32904 | |
| 14 | 7.98 | 215.7 | 13.6 | 12.7 | |
| 15 | 7.77 | 210 | 22.4 | 9.74 | |

### Example 4

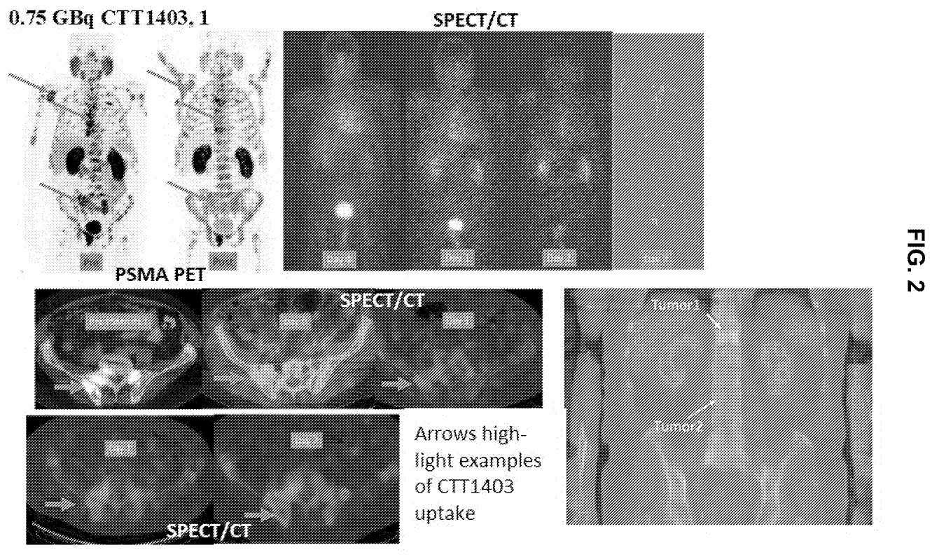

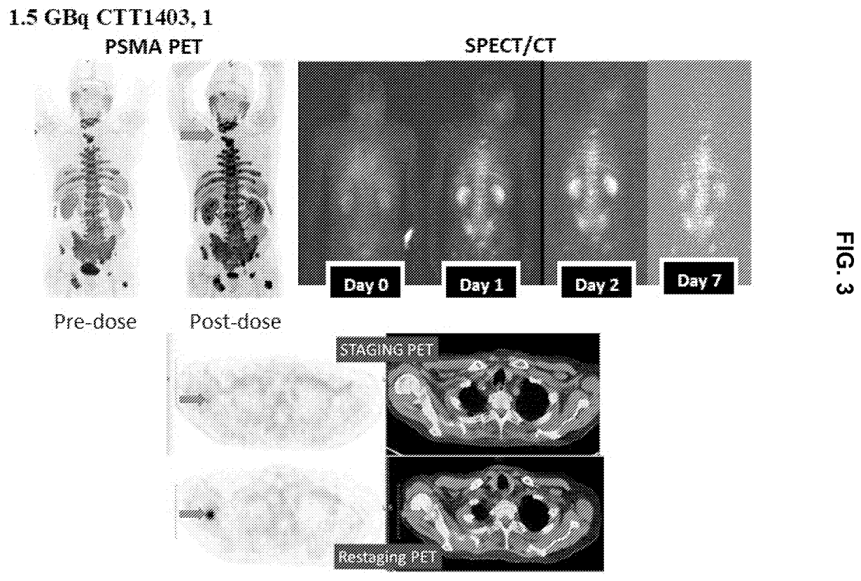

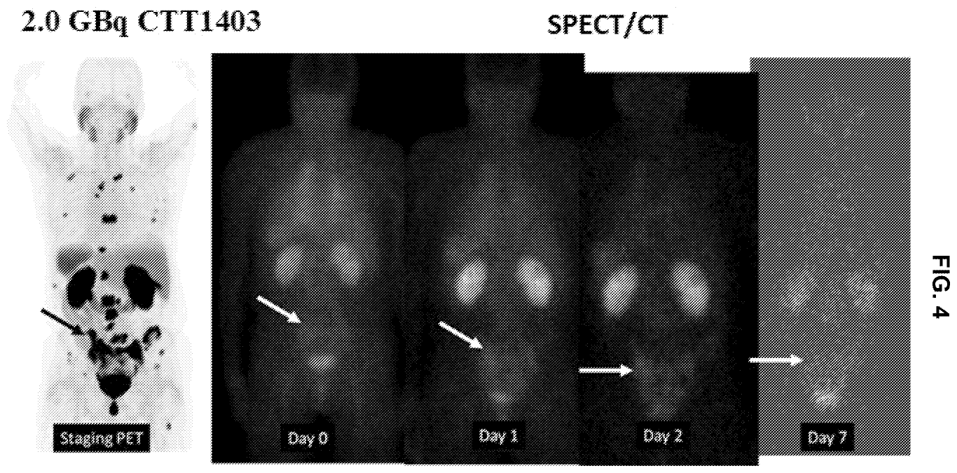

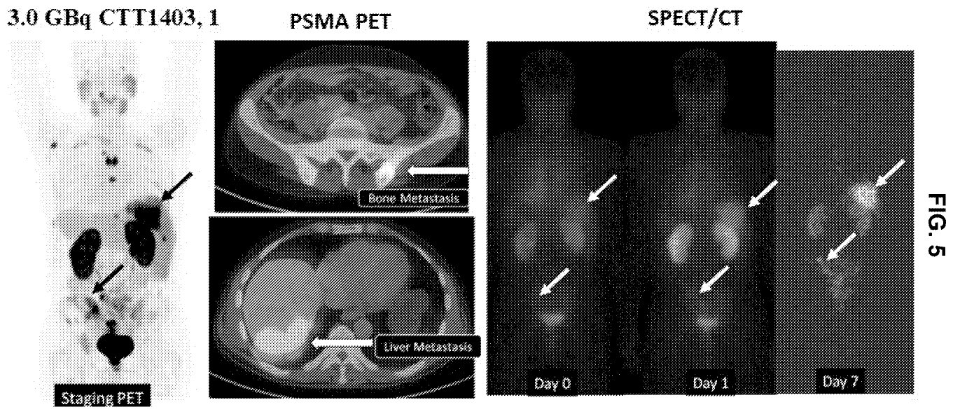

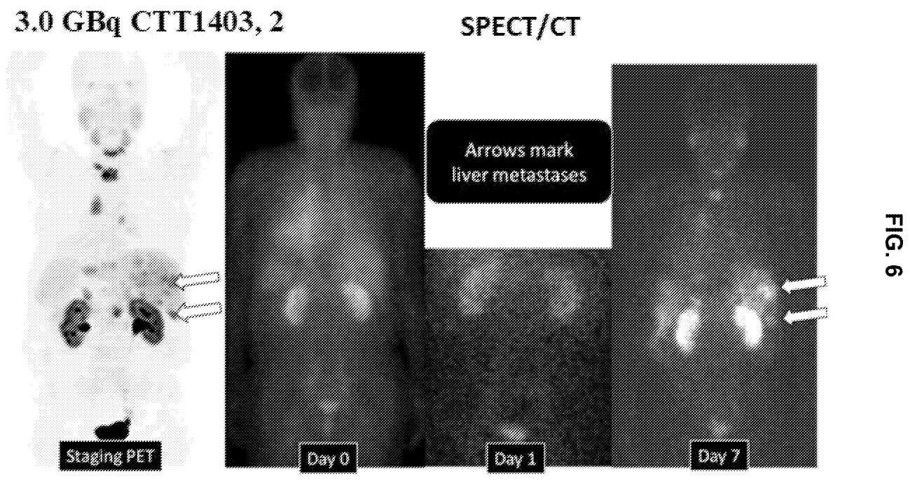

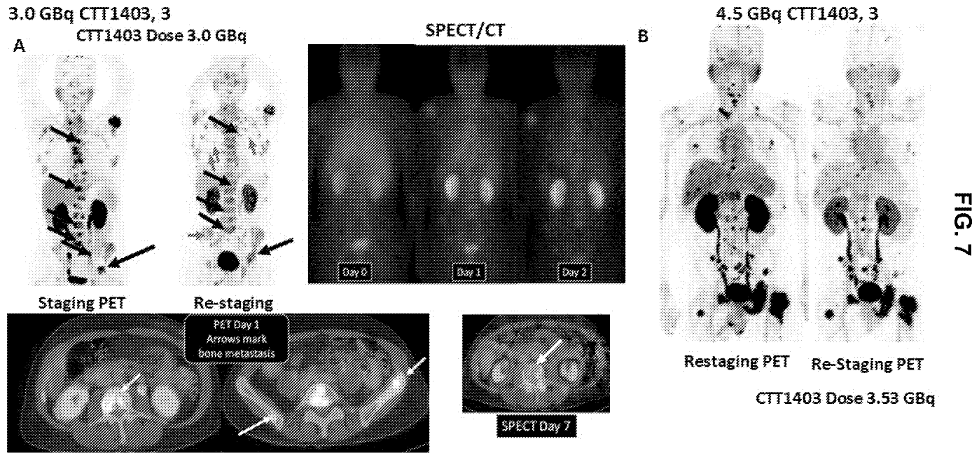

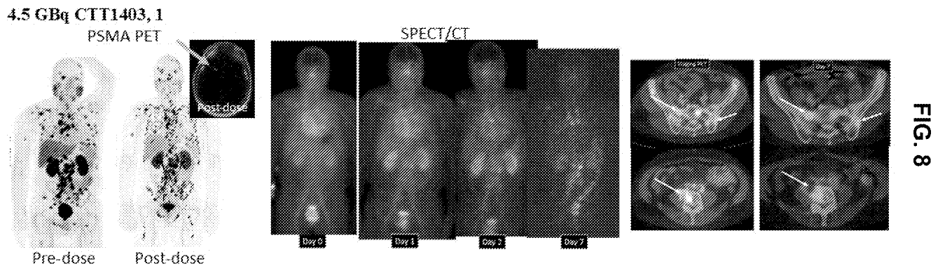

CTT1057 PSMA+ PET is used to screen patients for PSMA avid tumors prior to enrollment into the clinical trial. SPECT/CT images are collected serially on days 1, 3, 5 and 8 following CTT1403 dose administration to assess dosimetry, or exposure of normal organs and the tumor to the radionuclide, 177-lutetium. Patients who do not demonstrate disease progression or unacceptable toxicity following a single dose of CTT1403, are imaged again using standard MRI or CT scan and in some cases CTT1057 PET and offered a second dose of CTT1403 at the same dose level as the first they received. FIGS. 2-7 show the PET images collected prior to and after CTT1403 and serial SPECT/CT images collected from patients enrolled in the clinical trial. Further, Table 4 compares the doses in kidney and tumor reported by Violet et al (Violet, J., et al., “Dosimetry of (177)Lu-PSMA-617 in Metastatic Castration-Resistant Prostate Cancer: Correlations Between Pretherapeutic Imaging and Whole-Body Tumor Dosimetry with Treatment Outcomes” J Nucl Med, 2019. 60(4): p. 517-523) compared to findings to date in patients administered CTT1403. Early reported CTT1403 tumor dose for patients at 7.5 GBq+/−10% is at least similar and possibly even greater compared to ¹⁷⁷Lu-PSMA-617. This early achievement of comparable Gy dosage to tumors of interest is encouraging; based on these preliminary results, forthcoming data is expected to yield enhanced radioactive dosages. In particular, CTT1403 was found to significantly outperform 177Lu-PSMA-617 in Gy/GBq in patients with PSA<50% (i.e., where patients experienced less than a 50% reduction in circulating PSA).

TABLE 4

| | 177Lu-PSMA-617 | | CTT1403 | |
|---|---|---|---|---|
| | (7.4 GBq) | | (7.5 GBq +/− 10%) | |
| | Mean Dose | Dose | Mean Dose | Dose |
| | for (Gy) | Gy/GBq | (Gy) | Gy/GBq |
| Kidney dose | 3.2 | 0.77 | 4.895 | 0.689 |
| Tumor (PSA ≥ 50%) | 14.1 | 1.91 | | |
| Tumor (PSA < 50%) | 10.4 | 1.41 | | |
| Tumor Dose to date | | | 14.61* | 1.948* |

*Preliminary data from 2 tumors per patient in first 2 patients in cohort (patients 14 and 15).

### Example 5

Reduction in prostate specific antigen (PSA) at least 12 weeks after therapeutic administration is generally thought to be predictive of therapeutic efficacy. PSA levels at screening, at CTT1403 administration and at specified time points after CTT1403 administration are shown for selected patients enrolled in the trial, including patients still actively under observation (Patients 13, 14, 15). Reduction in PSA compared to Cycle 1, Day 1 has been observed in 4 patients to date: Patient 002 (75% maximum reduction), Patient 4 (40% maximum reduction), Patient 13 (14% reduction to date) and Patient 15 (9% reduction to date). To date, physical examination and monitoring have identified no serious adverse events after receipt of CTT1403. Table 5 shows the PSA levels measured at their intake screening and during their first and second courses of CTT1403 for patients that progressed at least that far in treatment, at the indicated date.

TABLE 5

| Patient | | | PSA | |
|---|---|---|---|---|
| ID | Visit | Visit Date | Result | Units |
| 2 | Cycle 1-Day 1 | 2019 Jun. 19 | 162.353 | ug/L |
| 2 | Cycle 1-Day 22 | 2019 Jul. 9 | 185.132 | ug/L |
| 2 | Cycle 2-Day 1 | 2019 Aug. 1 | 194.320 | ug/L |
| 2 | End of Treatment | 2019 Sep. 5 | 215.919 | ug/L |
| 2 | 4 Weeks Post Treatment | 2019 Oct. 4 | 405.6 | ug/L |
| 2 | 8 Weeks Post Treatment | 2019 Nov. 4 | 167.013 | ug/L |
| 2 | 12 Weeks Post Treatment | 2019 Dec. 2 | 45.716 | ug/L |
| 4 | Screening | 2019 Sep. 30 | 70.228 | ug/L |
| 4 | Cycle 1-Day 1 | 2019 Oct. 23 | 65.684 | ug/L |
| 4 | Cycle 1-Day 22 | 2019 Nov. 13 | 77.876 | ug/L |
| 4 | End of Treatment | 2019 Nov. 26 | 115.658 | ug/L |
| 4 | 8 Weeks Post Treatment | 2020 Jan. 20 | 41.80 | ug/L |
| 13 | Screening | 2020 Sep. 8 | 166.793 | ug/L |
| 13 | Cycle 1-Day 1 | 2020 Oct. 7 | 146.133 | ug/L |
| 13 | Cycle 1-Day 22 | 2020 Oct. 29 | 148.217 | ug/L |
| 13 | Cycle 2-Day 1 | 2020 Nov. 18 | 135.620 | ug/L |
| 13 | Cycle 2-Day 22 | 2020 Dec. 9 | 126.392 | ug/L |
| 13 | TBD (follow-up is ongoing) | | TBD | |
| 14 | Screening | 2020 Nov. 10 | 16.310 | ug/L |
| 14 | Cycle 1-Day 1 | 2020 Dec. 2 | 22.642 | ug/L |
| 14 | Cycle 1-Day 22 | 2020 Dec. 22 | 142 | ug/L |
| 14 | TBD (follow-up is ongoing) | | TBD | |
| 15 | Screening | 2020 Nov. 17 | 410.363 | ug/L |
| 15 | Cycle 1-Day 1 | 2020 Dec. 9 | 595.739 | ug/L |
| 15 | Cycle 1-Day 22 | 2020 Dec. 28 | 543.5 | ug/L |
| 15 | TBD (follow-up is ongoing) | | TBD | |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for treating cancer in a subject in need thereof, wherein the cancer has tumors that express PSMA or vasculature associated with the tumors expresses PSMA, the method comprising administering to the subject an effective amount of CTT1403, wherein CTT1403 is a compound having the formula: 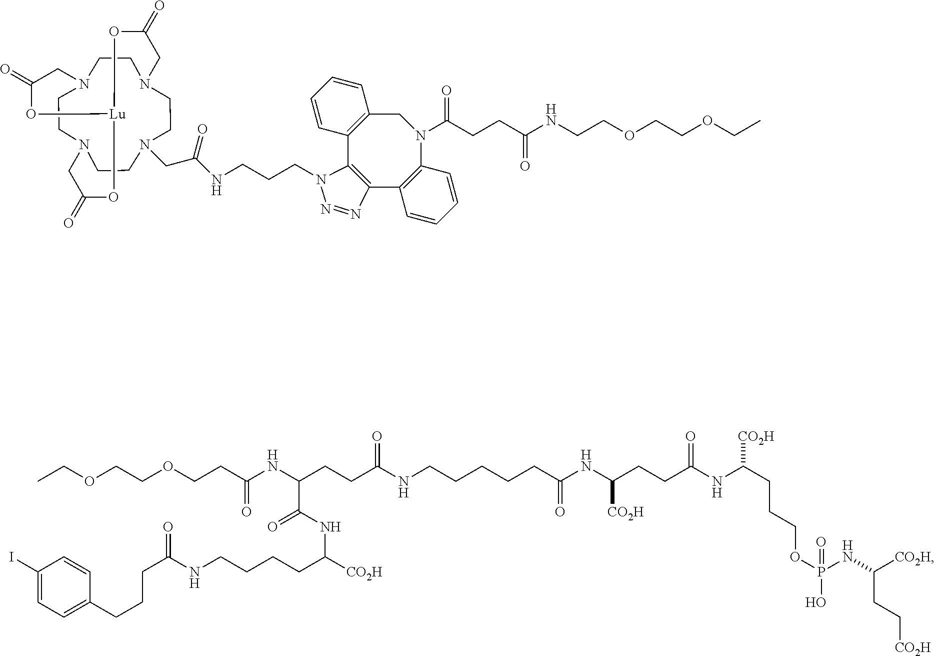
or a salt thereof, wherein the effective amount is about 0.75 GBq to about 14.0 GBq.

2. The method of claim 1, wherein the cancer is prostate cancer.

3. The method of claim 1, wherein CTT1403 or a salt thereof is delivered in a radiostabilizing solution.

4. (canceled)

5. (canceled)

6. The method of claim 1, further comprising administering an imaging agent to the subject.

7. (canceled)

8. (canceled)

9. (canceled)

10. The method of claim 6, wherein the imaging agent is CTT1057, wherein CTT1057 is: (((S)-4-carboxy-4-((S)-4-carboxy-4-(6-(4-(fluoro-¹⁸F)benzamido)hexanamido)butanamido)butoxy)(hydroxy)phosphoryl)-L-glutamic acid, or a salt thereof.

11. The method of claim 6, wherein the imaging agent is ⁶⁸Ga-PSMA-11.

12. The method of claim 6, wherein the imaging agent is DCFPyL F-18.

13. The method of claim 1, further comprising imaging the subject.

14. The method of claim 1, wherein CTT1403 or a salt thereof is administered on a dosage schedule, wherein 2, 3, 4, 5, 6, 7, or 8 doses are administered at an interval of from about 1 to 16 weeks between each dose.

15. The method of claim 14, wherein the interval between doses is about 3 to 12 weeks.

16. (canceled)

17. (canceled)

18. (canceled)

19. The method of claim 1, wherein the CTT1403 or a salt thereof is administered in an amount of about 0.75 GBq to about 10.0 GBq, e.g., about 0.75 GBq to about 9.0 GBq, or about 0.75 GBq to about 8.0 GBq, or about 0.75 GBq to about 7.0 GBq, or about 0.75 GBq to about 6.0 GBq, or about 0.75 GBq to about 5.0 GBq, or about 0.75 GBq to about 4.0 GBq, or about 0.75 GBq to about 3.5 GBq, or about 0.75 GBq to about 3.0 GBq, or about 0.75 GB to about 2.0 GBq, or about 0.75 GBq to about 1.5 GBq, or about 0.75 GBq to about 1 GBq.

20. (canceled)

21. (canceled)

22. The method of claim 1, wherein each effective amount of CTT1403 delivers at least 8 Gy of absorbed radiation to at least one cancer tumor.

23. (canceled)

24. The method of claim 1, wherein the cancer is prostate-specific membrane antigen positive (PSMA-positive or PSMA+),

25. The method of claim 1, wherein the cancer is castration resistant.

26. The method of claim 1, wherein the cancer is androgen.

27. The method of claim 1 wherein the cancer is organ specific or the cancer is localized, or the cancer is metastatic.

28. (canceled)

29. (canceled)

30. The method of claim 1, wherein the subject previously received an anti-androgen treatment.

31. The method of claim 1, wherein the subject is identified as having PSMA-positive cancer.

32. A method for treating prostate cancer in a subject in need thereof, the method comprising
identifying a subject as having PSMA-positive prostate cancer; and
administering to the subject a dose of about 0.75 GBg to about 14.0 GBg of CTT1403 or
a salt thereof, wherein CTT1403 is a compound having the formula

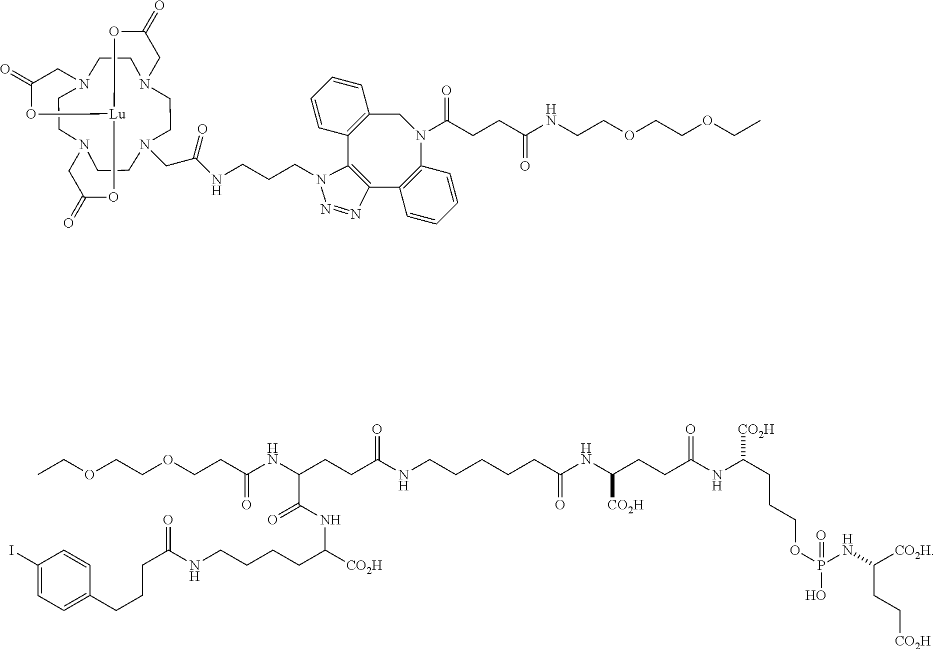

33-41. (canceled)

42. A method of administering at least about 10 Gy of absorbed radiation to the cancerous tissue of a patient being treated for cancer, comprising administering one or more doses of CTT1403 to the patient, where each dose comprises from about 6-12 GBq of radioactivity.

43-60. (canceled)
